(19) United States
(12) Patent Application Publication
(10) Pub. No.: US 2007/0009614 A1
(43) Pub. Date: Jan. 11, 2007

(54) Composition and method for treating hypertension

(75) Inventor: Gordon Stokes, St. Leonards (AU)

Correspondence Address: FOLEY & LARDNER LLP, P.O. BOX 80278, SAN DIEGO, CA, 92138-0278, US

(73) Assignee: Northern Sydney Area Health Service

(21) Appl. No.: 11/520,297

(22) Filed: Sep. 12, 2006

Related U.S. Application Data
(62) Division of application No. 10/255,447, filed on Sep. 25, 2002.

(30) Foreign Application Priority Data
May 16, 2002 (AU) PS2369

(51) Int. Cl. A61K 33/00; A61K 31/401; A61K 31/21

(52) U.S. Cl. 424/718; 514/423; 514/509

(57) ABSTRACT

The present invention relates to a composition for the treatment and/or prevention of hypertension, said composition comprising an synergistic anti-hypertensive combination of a therapeutically effective amount of at least one angiotensin II inhibitor, and a therapeutically effective amount of at least one nitric oxide donor; said composition optionally further comprising a pharmaceutically acceptable carrier, diluent and/or adjuvant.

### TECHNICAL FIELD

The present invention relates generally to a composition and method for treating and/or preventing hypertension. More particularly, the present invention relates to a composition and method for treating isolated systolic hypertension (ISH) and/or for treating systolic hypertension of the elderly (SHE).

### BACKGROUND OF THE INVENTION

Isolated systolic hypertension (ISH) and associated widening of pulse pressure have been identified as important risk factors for cardiovascular disease in the elderly,¹⁻⁴ and may persist despite the use of conventional antihypertensive drugs.⁵⁻⁶ ISH generally refers to cases where diastolic blood pressure without treatment is normal while systolic blood pressure is high. SHE, on the other hand, generally refers to cases where diastolic blood pressure is normalised by treatment while the systolic blood pressure remains high.

The research disclosed in the present application indicates that high systolic blood pressure in the elderly (such as, people greater than 65 years of age) is frequently associated with widened systemic and aortic pulse pressure caused by giant pulse-wave reflection. This wave reflection may signal endothelial dysfunction in conduit arteries.

Further, deficiency of endothelial nitric oxide (NO) production is a potential factor in the pathophysiology of increased arterial stiffness, which in turn leads to the exaggerated pulse wave reflection and wide pulse pressure typical of systolic hypertension of the elderly (SHE)⁷⁻⁸. Raised systolic blood pressure is an important cardiovascular risk factor in elderly persons and is often resistant to standard anti-hypertensive agents, including ACE inhibitors⁵. It is an attractive concept that SHE might be controlled best by therapy directed at redressing endothelial NO dysfunction. ACE inhibitors are known to regress structural changes in the endothelium of hypertensive patients, but there is disagreement as to whether they improve NO bioavailability⁹⁻¹¹.

As a result of these associated complications with ISH and SHE, these two forms of hypertension tend to be unresponsive, or insufficiently responsive, to conventional anti-hypertensive treatment regimes.

Accordingly, there is a need for a more effective anti-hypertensive treatment regime which responds to the widening pulse pressure associated with these forms of hypertension.

### OBJECT OF THE INVENTION

It is an object of the present invention to provide a composition and method of treatment and/or prevention of hypertension, in particular, ISH and SHE, that seeks to address the above-mentioned need.

### SUMMARY OF THE INVENTION

In a first aspect, the present invention provides a composition for the treatment and/or prevention of hypertension, said composition comprising an synergistic anti-hypertensive combination of a therapeutically effective amount of at least one angiotensin II inhibitor and a therapeutically effective amount of at least one nitric oxide donor.

Typically, the composition further comprises a pharmaceutically acceptable carrier, diluent and/or adjuvent.

Typically, the angiotensin II inhibitor is an angiotensin converting enzyme (ACE) inhibitor. More typically, the ACE inhibitor may be selected from the group consisting of captopril, enalapril, monopril, ramipril, cilazopril, fosinopril, perindopril, any other ACE inhibitor suitable for the treatment of hypertension, or combinations thereof. Even more typically, the ACE inhibitor is captopril.

In various embodiments, the angiotensin II inhibitor may also be an angiotensin II receptor antagonist. More typically, the angiotensin II receptor antagonist may be selected from the group consisting of eprosartan, candesartan, irbesartan, telmisartan, losartan, valsartan, any other angiotensin II receptor antagonist suitable for the treatment of hypertension, or combinations thereof Even more typically, the angiotensin II receptor antagonist is eprosartan.

In further embodiments, the composition of the first aspect contains more than one angiotensin II inhibitor, including combinations of ACE inhibitors and angiotensin II receptor antagonists.

Typically, the nitric oxide donor is a long-acting nitrate or an extended-release nitrate. More typically, the nitric oxide donor may be selected from the group consisting of isosorbide mononitrate (ISMN), isosorbide dinitrate, sinitrodil, any other nitric oxide donor suitable for the treatment of hypertension, or combinations thereof. Even more typically, the nitric oxide donor is ISMN.

In yet still further embodiments, the composition of the first aspect further comprises at least one of a range of other drugs suitable for the treatment of hypertension selected from the group consisting of beta-blockers, such as atenolol, metoprolol, carvedilol, calcium antagonists, such as, nifedipine, amlodipine, felodipine, diltiazem, diuretics, such as, hydrochlorothiazide, amiloride, indapamide, chlorthalidone, frusemide, any other drugs suitable for the treatment of hypertension, such as, clonidine, hydrallazine, methyl dopa, or combinations thereof.

Generally, for preferred embodiments of the composition of the first aspect, typically in the form of a combined tablet, the ranges of the nitric oxide donor, preferably ISMN, are 1-200 mg, more preferably 40-160mg, more preferably still 60 mg. Typically, the angiotensin II inhibitor, preferably Captopril, is present in ranges from 1-100 mg, more preferably 10-90 mg, more preferably still 25 mg.

Typically, the combined tablet is scored to make breakable into halves. Embodiments wherein those ranges and figures are doubled may also be appropriate for the combined tablet of preferred embodiments.

Other typical combinations for the composition of the first aspect include combinations (all given mane) of ISMN 60 mg/fosinopril 10 mg; ISMN 120 mg/fosinopril 20 mg; ISMN 60 mg/enalapril 10 mg; ISMN 120 mg/enalapril 20 mg; ISMN 60 mg/perindopril 4 mg; ISMN 120 mg/ perindopril 8 mg; ISMN 60 mg/ramipril 5 mg; ISMN 120 mg/ramipril 10 mg; ISMN 60 mg/irbesartan 150 mg; ISMN 120 mg/irbesartan 300 mg; ISMN 60 mg/telmisartan 40 mg; ISMN 120 mg/telmisartan 80 mg; ISMN 60 mg/candesartan 8 mg; ISMN 120 mg/candesartan 16 mg, ISMN 60 mg/eprosartan 300 mg; and ISMN 120 mg/eprosartan 600 mg.

The composition of the first aspect and its various embodiments described above may be used in the treatment of hypertension, and more preferably in the treatment of isolated systolic hypertension and/or systolic hypertension of the elderly (SHE). One preferred embodiment of the composition also has indications for preventing and/or decreasing the incidence of myocardial infarction and stroke, particularly in patients with hypertension.

In a second aspect, the present invention provides a method for the treatment and/or prevention of hypertension in a patient, said method comprising administering to the patient a therapeutically effective amount of a composition of the first aspect.

Typically, the method of the second aspect would be used for the treatment of isolated systolic hypertension or systolic hypertension of the elderly. In either case, the preferred regime for this method of treatment would be in the range of nitric oxide donor, preferably ISMN, 1-200 mg/day, more preferably 10-150 mg/day, more preferably still 30-120 mg/day with angiotensin II inhibitor, preferably captopril, 1-100 mg/day, more preferably 8-70 mg/day, more preferably still 12.5-50 mg/day. Preferably, doses are given once daily in the early morning.

Some other typical combinations of angiotensin II inhibitors for the method of the second aspect are described in an earlier paragraph discussing other typical combinations for the composition of the first aspect.

In a third aspect, the present invention provides the use of a composition of the first aspect for the manufacture of a medicament for the treatment and/or prevention of hypertension in a patient.

In a fourth aspect, the present invention provides a composition of the first aspect when used for the treatment and/or prevention of hypertension in a patient.

In a fifth aspect, the present invention provides a method of reducing a height of a pulse-wave reflection in a patient with hypertension, said method comprising administering to the patient a therapeutically effective amount of a composition of the first aspect.

In a sixth aspect, the present invention provides the use of a composition of the first aspect for the manufacture of a medicament for reducing a height of a pulse-wave reflection in a patient with hypertension.

In a seventh aspect, the present invention provides a composition of the first aspect when used for reducing a height of a pulse-wave reflection in a patient with hypertension.

In some preferred embodiments of the fifth to the seventh aspects of the invention, the height of the pulse-wave reflection is determined by applanation tonometry. In other embodiments, the height of the pulse-wave reflection is determined by inference from the finding that pulse pressure is consistently wide when measured by a standard office sphygmomanometer, or when measured by any other means capable of measuring the height to a sufficient degree of accuracy.

In a eighth aspect, the present invention provides a method of treating and/or preventing hypertension in a patient, said method comprising administering to a patient a combination of:

a therapeutically effective amount of at least one angiotensin II inhibitor; and

a therapeutically effective amount of at least one nitric oxide donor,

wherein said combination has an synergistic anti-hypertensive effect on the patient.

In a ninth aspect, the present invention provides the use of a combination of a therapeutically effective amount of at least one angiotensin II inhibitor and a therapeutically effective amount of at leat one nitric oxide donor, said combination having an synergistic anti-hypertensive effect, for the manufacture of a medicament for the treatment and/or prevention of hypertension in a patient.

In a tenth aspect, the present invention provides a combination of a therapeutically effective amount of at least one angiotensin II inhibitor and a therapeutically effective amount of at least one nitric oxide donor, said combination having an synergistic anti-hypertensive effect, when used for the treatment and/or prevention of hypertension in a patient.

In an eleventh aspect, the present invention provides a method of reducing a height of a pulse-wave reflection in a patient with hypertension, said method comprising administering to a patient a combination of

a therapeutically effective amount of at least one angiotensin II inhibitor; and

a therapeutically effective amount of at least one nitric oxide donor,

wherein said combination has an synergistic anti-hypertensive effect on the patient.

In a twelfth aspect, the present invention provides the use of a combination of a therapeutically effective amount of at least one angiotensin II inhibitor and a therapeutically effective amount of at leat one nitric oxide donor, said combination having an synergistic anti-hypertensive effect, for the manufacture of a medicament for reducing a height of a pulse-wave reflection in a patient with hypertension.

In a thirteenth aspect, the present invention provides a combination of a therapeutically effective amount of at least one angiotensin II inhibitor and a therapeutically effective amount of at least one nitric oxide donor, said combination having an synergistic anti-hypertensive effect, when used for reducing a height of a pulse-wave reflection in a patient with hypertension.

In some preferred embodiments of the eleventh to the thirteenth aspects of the invention, the height of the pulse-wave reflection is determined by applanation tonometry. In other embodiments, the height of the pulse-wave reflection is determined by inference from the finding that pulse pressure is consistently wide when measured by a standard office sphygmomanometer, or when measured by any other means capable of measuring the height to a sufficient degree of accuracy.

Typically, having regard particularly to the eighth to thirteenth aspects of the invention, the angiotensin II inhibitor and nitric oxide donor are administered to the patient simultaneously.

Also included within the scope of the invention are prodrugs. Typically prodrugs will be functional derivatives, such as ISMN. ISMN undergoes enzymatic degradation in the vascular smooth muscle to form nitric oxide, which acts through cyclic GMP-mediated processes to produce endothelium-independent vasodilatation in muscular arteries. Typical procedures for the selection and preparation of prodrugs are known to those of skill in the art and are described, for instance, in H. Bundgaard (Ed), Design of Prodrugs, Elsevier, 1985.

### BRIEF DESCRIPTION OF THE DRAWINGS

A preferred form of the present invention will now be described by way of example with reference to the accompanying figures wherein:

FIG. 1 illustrates mean values and SEM in Group I (no baseline angiotensin II inhibition) for aortic systolic blood pressure and augmentation index in relation to ingestion at 08.00 h on different study days of placebo, eprosartan 600 mg, captopril 25 mg, and isosorbide mononitrate (ISMN) 60 mg. (For eprosartan, n=10; for other three agents, n=11).

FIG. 2 illustrates mean values and SEM (n=6) for aortic systolic blood pressure and augmentation index in Group II (with AII inhibition at baseline) in relation to ingestion at 08.00 h on different study days of placebo and isosorbide mononitrate, 60 mg.

### DEFINITIONS

For the purposes of the present application the term “extended release” means contained in a matrix, or combined with excipients, which delay the release of and thereby prolong the duration of action of the active constituent.

Further, “long acting” means having a longer time of elimination (t one-half) from the plasma compartment than other drugs of the same class.

A “therapeutically effective amount”, as used herein, includes within its meaning a non-toxic but sufficient amount of the particular drug to which it is referring to provide the desired therapeutic effect. The exact amount required will vary from subject to subject depending on factors such as the patient's general health, the patient's age, the severity of systolic blood pressure elevation, the previous dose titration to the level of optimal response.

An “angiotensin II inhibitor”, as used herein, includes an angiotensin converting enzyme (ACE) inhibitor and/or an angiotensin II receptor antagonist.

In the context of this specification, the term “simultaneously” when referring to simultaneous administration of the relevant drugs means at exactly the same time, as would be the case, for example in embodiments where the drugs are combined in a single preparation. In other embodiments, “simultaneously” can mean one drug taken a short duration after another, wherein “a short duration” means a duration which allows the drugs to have their intended synergistic anti-hypertensive effect. Typically, a short duration for preferred embodiments of the invention would be up to and including 12 hours.

In the context of this specification, the term “comprising” means “including principally, but not necessarily solely”. Furthermore, variations of the word “comprising”, such as “comprise” and “comprises”, have correspondingly varied meanings.

### BEST MODE OF PERFORMING THE INVENTION

The high pulse pressure of ISH is usually associated with the presence in the aortic pulse wave of a prominent reflection peak, which combines with the tail of the incident peak arising from cardiac ejection to increase pulse pressure.¹²

We have compared the antihypertensive activity and effects on pulse wave contour of single doses of ISMN, captopril and eprosartan in a group of nitrate-naive elderly patients with systolic hypertension. In a similar group, responses to ISMN were studied also in the presence of chronic treatment with angiotensin II inhibitors.

The pulse wave reflection associated with high systolic blood pressure in the elderly is sensitive to the effect of isosorbide mononitrate (ISMN), which reduces the height of reflection to a greater extent than conventional anti-hypertensive agents.

The effect of ISMN is selective in at least two respects: (1) it produces a much greater fall in systolic pressure than in diastolic pressure; and (2) it results in a fall in P2 (reflection or augmented pressure) which is greater than that in P1 (ejection peak), and in this respect, the nitrate (or nitric oxide donor) differs from angiotensin II inhibitory agents.

These properties of ISMN indicate that its hypotensive action is endothelium-independent. In contrast, the effects of angiotensin II inhibitors depend upon the endothelium being intact.

Put more succinctly, AII inhibitors reduce diastolic BP well in all forms of hypertension, and systolic BP in most, but fail to sufficiently lower systolic BP in SHE. This failure relates to lack of effect on pulse wave reflection (PWR).

ISMN has little effect on the diastolic BP in SHE, but decreases systolic BP by decreasing PWR and thereby systolic BP. ISMN has no known direct effect on the endothelium, but produces beneficial BP lowering and improvement in cardiac function through nitric oxide donation.

Moreover, the research disclosed herein has surprisingly indicated that a combination of at least one nitric oxide donor, preferably isosorbide mononitrate (ISMN), and at least one angiotensin II inhibitor, preferably an ACE inhibitor, such as captopril, and/or an angiotensin II receptor antagonist, preferably eprosartan, has an synergistic anti-hypertensive effect in patients with hypertension, and more particularly in patients over 65 years of age with systolic hypertension of the elderly, and/or patients with isolated systolic hypertension.

Accordingly, in more preferred embodiments, the present invention provides a synergistic anti-hypertensive combination as described in the previous paragraph, and as discussed in more detail below.

Pharmaceutical and Therapeutic Formulations

The pharmaceutical compositions of the present invention may be administered therapeutically. In a therapeutic application, compositions are administered to a patient already suffering from a disease, in an amount sufficient to cure or at least partially arrest the disease and its complications. Single or multiple administrations of the pharmaceutical compositions can be carried out with dose levels and pattern being selected by the treating physician.

The therapeutically effective dose level for any particular patient will depend upon a variety of factors including: the disorder being treated and the severity of the disorder; the composition employed; the age, body weight, general health, sex and diet of the patient; the time of administration; the route of administration; the duration of the treatment; drugs used in combination or coincidental with the synergistic composition, together with other related factors well known in medicine.

One skilled in the art would be able, by routine experimentation, to determine an effective, non-toxic amount of this treatment regime which would be required to treat the disorders and diseases to which the synergistic combination of the present invention is applicable.

The preferred regime for this method of treatment would be ISMN 30-120 mg/day with Captopril 12.5-50 mg/day. Doses given once daily in the early morning.

Other typical combinations for the method include combinations (all given mane) of ISMN 60 mg/fosinopril 10 mg; ISMN 120 mg/fosinopril 20 mg; ISMN 60 mg /enalapril 10 mg; ISMN 120 mg/enalapril 20 mg; ISMN 60 mg/perindopril 4 mg; ISMN 120 mg/perindopril 8 mg; ISMN 60 mg/ramipril 5 mg; ISMN 120 mg/ramipril 10 mg; ISMN 60 mg/irbesartan 150 mg; ISMN 120 mg/irbesartan 300 mg; ISMN 60 mg/telmisartan 40 mg; ISMN 120 mg/telmisartan 80 mg; ISMN 60 mg/candesartan 8 mg; ISMN 120 mg/candesartan 16 mg; ISMN 60 mg/eprosartan 300 mg; and ISMN 120 mg/eprosartan 600 mg.

For embodiments, in which the composition of the first aspect is used as for example in a combined tablet, the combinations are preferably ISMN 60 mg/captopril 25 mg, scored to make breakable into halves, and ISMN 120 mg/captopril 50 mg. Further, the other typical combinations of the previous paragraph may also be suitable for various embodiments of compositions of the first aspect.

Further, it will be apparent to one of ordinary skill in the art that the optimal quantity and spacing of individual dosages of the composition of the present invention will be determined by the nature and extent of the condition being treated, the form, route and site of administration, and the nature of the particular vertebrate being treated. Also, such optimum conditions can be determined by conventional techniques.

It will also be apparent to one of ordinary skill in the art that the optimal course of treatment, such as, the number of doses of the composition of the present invention given per day for a defined number of days, can be ascertained by those skilled in the art using conventional course of treatment determination tests.

In general pharmaceutical formulations of the present invention may be prepared according to methods which are known to those of ordinary skill in the art and accordingly may include a pharmaceutically acceptable carrier, diluent and/or adjuvant.

These formulations are preferably administered by the oral route.

The carriers, diluents and adjuvants must be “acceptable” in terms of being compatible with the other ingredients of the formulation, and not deleterious to the recipient thereof.

Examples of pharmaceutically acceptable carriers or diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

The pharmaceutical compositions of the invention may be in the form of a composition in a form suitable for administration by oral ingestion (such as capsules, tablets, caplets, elixirs).

Some examples of suitable carriers, diluents, excipients and adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration.

Adjuvants typically include emollients, emulsifiers, thickening agents, preservatives, bactericides and buffering agents.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, steartic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may farther comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

The emulsions for oral administration may further comprise one or more emulsifying agents. Suitable emulsifying agents include dispersing agents as exemplified above or natural gums such as guar gum, gum acacia or gum tragacanth.

Pharmaceutical compositions of the present invention may be prepared by blending, grinding, homogenising, suspending, dissolving, emulsifying, dispersing and/or mixing the angiotensin II inhibitor(s) and the nitric oxide donor(s), with the selected excipient(s), carrier(s), adjuvant(s) and/or diluent(s).

One type of pharmaceutical composition of the present invention in the form of a tablet or capsule may be prepared by (a) preparing a first tablet or a capsule comprising at least one of the active substances together with any desired excipient(s), carrier(s), adjuvant(s) and/or diluent(s), and (b) preparing a second tablet or a capsule, wherein the second tablet or the capsule includes the remaining active substance(s) and the first tablet or capsule.

Another type of pharmaceutical composition of the present invention in the form of a capsule may be prepared by (a) preparing a first capsule comprising at least one of the active substances together with any desired excipient(s), carrier(s), adjuvant(s) and/or diluent(s), and (b) preparing a second capsule, wherein the second capsule includes the remaining active substance(s) and the first capsule.

A further type of pharmaceutical composition of the present invention in the form of a tablet may be prepared by (a) preparing a capsule comprising at least one of the active substances together with any desired excipient(s), carrier(s), adjuvant(s) and/or diluent(s), and (b) preparing a tablet, wherein the tablet includes the remaining active substance(s) and the capsule.

The invention will now be described in greater detail by reference to specific Examples, which should not be construed as in any way limiting the scope of the invention.

### EXAMPLES

### Example 1

Comparison of Isosorbide Mononitrate, Captopril and Eprosartan Effect on Pulse Wave Reflection

Subjects and Methods

The subjects were sixteen patients with long-standing hypertension, referred from their family physicians after having undergone treatment trials using a variety of conventional antihypertensive agents without reaching satisfactory control of systolic blood pressure. One subject had a history of unilateral renal artery stenosis successfully treated by angioplasty; in the remainder causes of secondary hypertension had been excluded by routine screening tests. Plasma creatinine concentration was <0.15 mmol/l in every case. There was a history of myocardial infarction Or coronary bypass grafting in eight subjects, of peripheral vascular disease in four, and of diabetes mellitus (not requiring medication) in one. All patients were fully ambulant without symptomatic cardiac disease or known vascular aneurysm. Their current regimen of antihypertensive therapy had been stable for three weeks or more, and was continued unchanged through the present study. None had received nitrate therapy previously. Two different study protocols were used. One patient participated in both protocols with a 15-month break in between. Entry to either study required that systolic blood pressure should be between 150 and 200 mmHg, and that diastolic blood pressure should be equal to or less than 100 mmHg.

Group I protocol. A double-blind randomised crossover study of three drugs and placebo was carried out in eleven of the subjects, five men and six women, 59-82 (mean 69.8) years of age. Their baseline anti-hypertensive therapy, which excluded ACE inhibitors and angiotensin II receptor antagonists, consisted of one to three of the following drugs in conventional dosage:. diuretics (7 cases), beta blockers (7), prazosin (1), amlodipine (4), nifedipine controlled-release formulation (2). The study medication was administered at 0805 h; encapsulated single doses of placebo, ISMN 60 mg (extended-release preparation, AstraZeneca, Australia), eprosartan 600 mg and captopril 25 mg were given separately in random order on four study days each separated from the next by one to two weeks. One subject did not complete the eprosartan phase. Duplicate observations of brachial blood pressure, standing and seated, were made every 60 min from 0800 h to 1600 h on each study day. The pulse wave and pulse rate were measured at the same intervals. Between observations, the subjects engaged in sedentary recreational activities in a temperature-controlled environment. A light meal was given at 12.30 h.

Group II protocol. Another group of six patients, three men and three women, 59-81 (mean 72.8) years of age, had a randomised double-blind crossover study in which ISMN 60 mg was given at 0800 h on one day, and placebo at 0800 h on the other. The two study days were one to two weeks apart. Observations were made from 0800 h to 1600 h as in Group I. These subjects differed from Group I in that they were receiving treatment at study entry with ACE inhibitor or angiotensin II receptor antagonist drugs, as follows: monopril 10 mg/d (1 case), captopril 50 mg/d (1), ramipril 10 mg/day (1), ramipril 10 mg/d and candesartan 16 mg/d (1), irbesartan 300 mg/d (1), telmisartan 40 mg/d (1). Their other baseline antihypertensive drug therapy consisted of 1-2 of the following agents in conventional dosage: hydrochlorthiazide (1), beta blockers (2), amlodipine (1), diltiazem controlled-delivery formulation (2).

Brachial blood pressure was recorded by sphygmomanometer, and pulse wave tonometry was performed at the radial artery with the patient seated. The aortic pulse wave form was determined and the aortic first peak pressure (P1) and augmentation pressure (P2) were quantified by computer software (SphygmoCor, AtCor Medical, Sydney), as previously reported.¹³ Augmentation index (P2 expressed as % of pulse pressure) described the magnitude of wave reflection. Statistical analysis was by repeated measures analysis of variance using PRISM (GraphPad Software Inc., San Diego, Calif.) and post-hoc paired t tests. Values given are mean±SEM.

Results

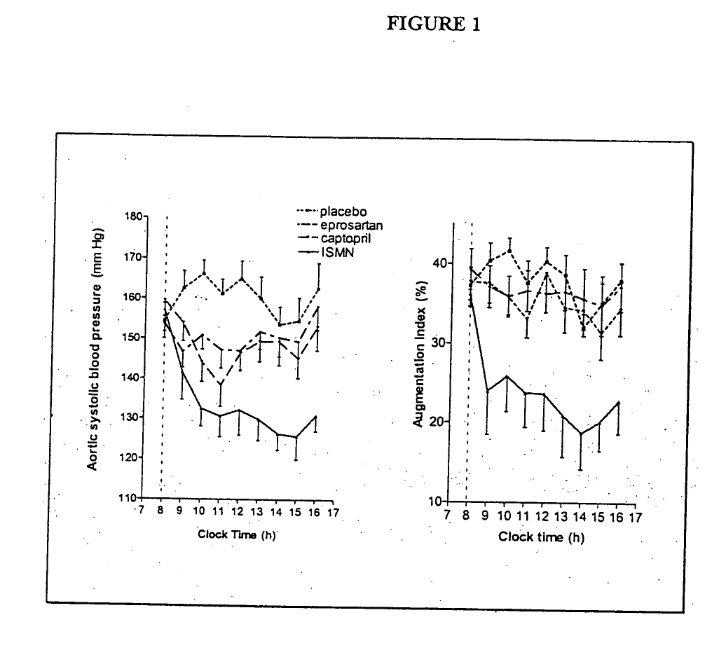

The effects in Group I of single doses of ISMN, captopril and eprosartan on aortic systolic blood pressure and augmentation index are shown in FIG. 1, and those on brachial blood pressure and heart rate are shown in Table 1. All three agents significantly decreased aortic systolic blood pressure (for ISMN and captopril, P<0.0001; for eprosartan, P<0.001), and this effect was greater with ISMN than with captopril or eprosartan (P<0.0001). At the respective nadirs of hypotensive effect for the three agents, the aortic systolic pressure was lower than control by 34 mm Hg (P<0.001) with ISMN (10.00 h), by 23 mm Hg (P<0.001) with captopril (11.00 h), and by 15 mm Hg (P<0.05) with eprosartan (09.00 h).

Separation of the effect on aortic pulse pressure into P1 and P2 components showed that eprosartan, captopril and ISMN each produced small decreases in P1 (at nadir 4±2, 9±2 and 11±2 mmHg, respectively) which were not significantly different between agents, but the decrease in P2 was significantly greater (P<0.0001) for ISMN (at nadir, 19±3 mm Hg) than for captopril (9±3 mm Hg) or eprosartan (6±3 mm Hg). Neither captopril nor eprosartan altered augmentation index, but with ISMN it was significantly reduced (P<0.0001) throughout the post-dose observation period (see FIG. 1).

All three agents significantly decreased sitting and standing systolic brachial blood pressure (P<0.005) without appreciable orthostatic effect (see Table 1). Heart rate showed an average post-dose increase of 3±1 b.p.m. (P<0.001) with ISMN, but was not changed by captopril or eprosartan. For ISMN, the falls in systolic pressure values were much greater than those in diastolic pressure and were more prolonged than the changes in systolic pressure observed with the other two agents; time trends were similar to those for aortic systolic blood pressure (see FIG. 1).

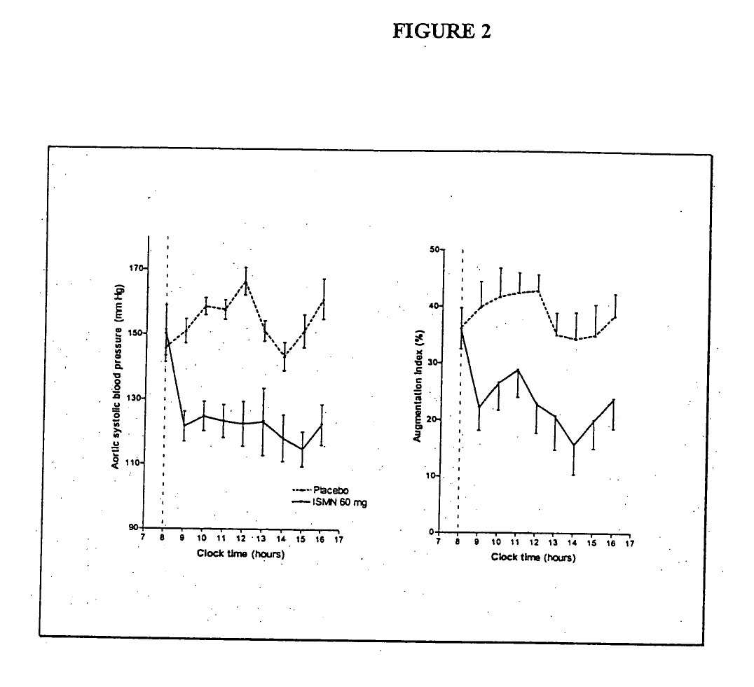

FIG. 2 shows the effects of single doses of ISMN on aortic systolic blood pressure and augmentation index for Group II (six patients with baseline therapy that included AII inhibition). The mean control values (placebo day, 08.00 h to 16.00 h) for aortic systolic blood pressure and augmentation index were 154±2 mm Hg and 38.5±1.1%, respectively. In comparison the corresponding values for Group I were to 160±2 mm Hg and 37.9±1.0%. Both aortic systolic pressure and augmentation index decreased significantly with ISMN in Group II (P<0.0001); the average post-dose decreases were, respectively, 34±3 mmHg and 16±1%, in comparison to decreases of 29±2 mmHg and 15±1% in Group I. Sitting and standing brachial systolic blood pressure in Group II were each decreased by ISMN (P<0.0001). Sitting brachial systolic blood pressure decreased from control values of 176 and 173 mm Hg at 12.00 h and 16.00 h, by 37 and 34 mm Hg (each P<0.001), respectively. Corresponding decreases for sitting diastolic pressure (13 and 10 mm Hg), and for standing systolic and diastolic blood pressure, were not significant at these time points. Heart rate was increased overall from a mean value of 60 to 64 bpm (P <0.001).

Discussion

ISMN reduced systolic blood pressure strongly in these nitrate-naive ISH patients, with minor effects on diastolic blood pressure and heart rate. Studies with nitroglycerin infusion have shown an accentuated hypotensive response during standing¹⁴: in our study, there was a mild orthostatic effect with ISMN, which was not greater than with the other two agents given. The decrease in aortic systolic pressure with ISMN was due partly to a reduction in amplitude of the exaggerated pulse wave reflection found in these patients with ISH, as indicated by a peak decrease in augmentation index of approximately 50% of control (see FIGS. 1 and 2),

The effects on systolic pressure were greater for ISMN than for captopril or eprosartan in the doses given, which were in the mid-range of dosage recommended for routine clinical therapy. The depressor effect of each agent in relation to placebo reached nadir 1-3 hours after dosing, within the time to reach peak plasma concentration reported for ISMN (3-4 h ¹⁵) and eprosartan (1-2 h ¹⁶) but slightly later than that reported for captopril (1 h ¹⁷). The effect of ISMN (which was in an extended-release preparation) was sustained through the remainder of the observation period; that of the other agents diminished consistent with their shorter elimination half-lives. ^{15, 17}

The present study was short-term and single-dose, whereas a proper evaluation of relative efficacy of the three agents would require dose-response analysis at steady state after continued dosing. This applies particularly in the case of eprosartan, postulated to reduce sympathetic tone by a pre-synaptic action;¹⁸ such an action could require changes in neurotransmitter release needing time to develop. Chronicity of therapy would be required also with ACE inhibitor use, to allow functional and structural changes in the endothelium¹⁰ that could influence the magnitude of aortic systolic or augmentation pressure.¹⁹

However, qualitative comparisons could be made between the short-term effects of ISMN, captopril and eprosartan on aortic pulse wave contour. ISMN produced a fall in P1 (first peak pressure) similar to that observed with captopril or eprosartan, but a greater fall in P2 (augmentation pressure) than did these agents. Integration of these data as augmentation index (FIG. 1, right panel) renders the marked difference in the effects of the drugs on wave reflection more evident.

It has been suggested that augmentation index may indicate the extent of endothelial dysfunction in vivo.^{19, 20} Such dysfunction is known to occur in essential hypertension,^{21,22} and was suspected in the present patient series because of high augmentation index, a history of long-standing hypertension, and a background of known vasculopathy in 8 of the 17 cases. AII inhibitor therapy has been reported to improve endothelial dysfunctions¹⁸ and decrease wave reflection,²³ so it was of interest to determine whether the chronic AII inhibition of Group II resulted in any overlap of effect on wave reflection with that of ISMN. It was found that the fall in augmentation index resulting from ISMN in Group II was commensurate with that in Group I. Thus, ISMN appeared to correct the process giving rise to magnified wave reflection in ISH by an effect distinct from that exercised by either acute or chronic AII inhibition. Indeed, of the antihypertensive agents used in treatment of this selected series of patients, both at baseline and within the study period, ISMN was shown to be uniquely effective in decreasing wave reflection.

ISMN is a pro-drug that undergoes enzymatic degradation in the vascular smooth muscle cell to form nitric oxide (NO), which acts through cyclic GMP-mediated processes to produce endothelium-independent vasodilatation in muscular arteries. The level of the arterial tree at which the effect of NO donors on wave reflection is operative has been a subject of some controversy. In the normal circulation, vasorelaxation after giving nitrates is greater in conduit arteries than in major central arteries or in arterioles. Moderate changes in brachial artery compliance and total peripheral resistance have been shown in healthy volunteers with the NO donors, isosorbide dinitrate and sinitrodil.²⁴ However, in hypertensive cardiovascular disease the muscle layer of larger arteries is progressively replaced by collagen, while the muscularis of small arteries becomes hypertrophied. These developments may tend to shift the foci of reflection distally to the smaller conduit arteries and arterioles, where nitrate-induced dilatation could produce relatively larger changes in peripheral resistance.

We have speculated that shear stress generated by each pulse wave excites release of a spurt of endothelial NO causing transient vasodilatation in muscular arteries: this allows forward progression of the pulse volume more distally into vascular beds, and limits the amplitude of wave reflection. We postulate that with endothelial dysfunction, pulsatile NO production is impaired. The pulse wave impacts a tonically constricted small artery network; the resulting recoil induces an amplified wave reflection, which is a major contributor to the expanded pulse pressure of ISH. NO donors such as ISMN diminish the recoil and modulate wave reflection. However, with antihypertensive drugs that require endothelial NO production to mediate their actions, a beneficial effect on wave reflection is delayed until endothelial recovery has occurred.

TABLE 1
Effects on systolic and diastolic blood pressure, sitting and standing, and on
heart rate (sitting) at 11.00 h of single doses of placebo, isosorbide mononitrate
(ISMN) 60 mg, captopril 25 mg, and eprosartan 600 mg, given at 08.00 h.

| | Sitting brachial blood pressure | | Standing brachial blood pressure | | |
|---|---|---|---|---|---|
| Study drug | systolic | diastolic | systolic | diastolic | Heart rate |
| Placebo | 173 ± 3 | 82 ± 3 | 166 ± 4 | 83 ± 3 | 60 ± 4 |
| ISMN | 145 ± 5* | 77 ± 3 | 139 ± 4* | 77 ± 3 | 60 ± 5 |
| Captopril | 148 ± 5* | 78 ± 2 | 143 ± 5* | 78 ± 2 | 59 ± 4 |
| Eprosartan | 158 ± 5 | 78 ± 3 | 152 ± 4 | 83 ± 4 | 59 ± 4 |

*P < 0.01 (Bonferroni post-test).
Values are mean (SEM). Units for blood pressure are mm Hg, and for heart rate are bpm.

### Example 2

Capsule Composition

A pharmaceutical composition of the invention in the form of a capsule may be prepared by filling a standard two-piece hard gelatin capsule with 60 mg of ISMN and 25 mg of captopril, in powdered form, 100 mg of lactose, 35 mg of talc and 10 mg of magnesium stearate.

### REFERENCES

1. SHEP Cooperative Research Group. Prevention of stroke by antihypertensive drug treatment in older persons with isolated systolic hypertension: final results of the Systolic Hypertension in the Elderly Program (SHEP). JAMA 1991;265:3255-3264.
2. Dahlof B, Lindholm L H, Hansson L, Schersten B, Ekbom T, Webster P-O. Morbidity and mortality in the Swedish Trial in Old Patients with Hypertension (Stop-Hypertension). Lancet 1991; 338: 1281-1285.
3. Staessen J A, Gasowski J, Wang J G, Thijs L, Den Hond E, Bissel J P, Coope J, Ekbom T, Gueuffier F, Liu L, Kerlikowske K, Pocock S, Fagard R H. Risks of untreated and treated isolated systolic hypertension in the elderly: meta-analysis of outcome trials. Lancet 2000;355:865-872.
4. Franklin S S, Khan S A, Wong N D, Larson M G, Levy D. Is pulse pressure useful in predicting risk for coronary heart disease? The Framingham Heart Study. Circulation 1999;100:354-360.
5. Morgan T O, Anderson A I E, MacInnes R J. ACE inhibitors, beta-blockers, calcium blockers, and diuretics for the control of systolic hypertension, Amer J Hypertens 2001;14:241-247.
6. Mancia G. Giannattasio C, Failla M, Sega R, Parati G. Systolic blood pressure and pulse pressure: role of 24-h mean values and variability in the determination of organ damage. J Hypertens 1999;17(suppl 5):S55-S61.
7. Cockcroft J R, Webb D J, Wilkinson I B. Arterial stiffness, hypertension and diabetes mellitus. J Hum Hypertension 2000; 14:377-380.
8. Blacher J, Staessen J A, Girerd X, Gasowski J, Thijs L, Liu L, et al. Pulse pressure not mean pressure determines cardiovascular risk in older hypertensive patients. Arch Intern Med 2000; 160:1085-1089.
9. Creager M A, Roddy M A. Effect of captopril and enalapril on endothelial function in hypertensive patients. Hypertension 1994; 24:499-505.
10. Kiowski W, Linder L, Nuesch R, Martina B. Effect of cilazopril on vascular structure and function in essential hypertension. Hypertension 1996; 27:371-376.
11. Taddei S, Virdis A, Ghiadoni L, Mattei P, Salvetti A. Effects of angiotensin converting enzyme inhibition on endothelium-dependent vasodilation in essential hypertensive patients. J Hypertens 1998; 16:447-456.
12. Black H R, Kuller L H, O'Rourke M F, Weber M A, Alderman M H, Benetos A, Burnett J, Cohn J N, Franklin S S, Mancia G, Safar M, Zanchetti A. The first report of the Systolic and Pulse Pressure (SYPP) Working Group. J Hypertens 1999;17(suppl 5):S3-S14.
13. Murrell W. Nitroglycerine as a remedy for angina pectoris. Lancet 1879;80:80-220.
14. Christenson B, Nordenfelt I, Westling H, White T. Intravenous infusion of nitroglycerin in normal subjects. Scand J Clin Lab Med 1989; 23:49-53.
15. Nyberg G. Current status of isosorbide-5-mononitrate therapy in chronic stable angina pectoris. Am J Therap 1994; 1:93-101.
16. Bottorff M B, Tenero D M. Pharmacokinetics of eprosartan in healthy subjects, patients with hypertension, and special populations. Pharmacotherapy 199; 19:73S-78S.
17. Duchin K L, Singhvi S M, Willard D A, Migdalof B H, McKinstry D N. Captopril kinetics. Clin Pharmacol Ther 1982;31:452-458.
18. Ohlstein E H, Brooks D P, Feuerstein G Z, Ruffolo R R. Inhibition of sympathetic outflow by the angiotensin II receptor antagonist, eprosartan, but not by losartan, valsartan or irbesartan: relationship to differences in prejunctional angiotensin II receptor blockade. Pharmacology 1997;55:244-251.
19. Wilkinson I B, Hall I R, MacCallum H, McKenzie I S, McEniery C M, van der Arend B J et al. Pulse-wavc analysis. Clinical evaluation of a non-invasive, widely applicable method for assessing endothelial function. Arterioscler Thromb Vasc Biol 2002; 22:147-152.Cohn J N. Vascular wall function as a risk marker for cardiovascular disease. J Hypertens 1999;17(suppl 5);S41-S44.
21. Panza J A. Quyyumi A A, Brush J E, Epstein S E. Abnormal endothelium-dependent vascular relaxation in patients with essential hypertension. N Engl J Med 1990; 323: 22-27.
22. Heistad D D, Armstrong M L, Baumbach G L, Faraci F M. Sick vessel syndrome. Recovery of atherosclerotic and hypertensive vessels. Hypertension 1995; 26: 509-513.
23. Chen C-H, Ting C-T, Lin S-J, Hsu T-L et al, Different effects of fosinopril and atenolol on wave reflections in hypertensive patients. Hypertension 1995; 90:1034-1041.
24. Van Bortel L M, Spek J J, Balkestein E J, Sardina M, Struijker H A. Is it possible to develop drugs that act more selectively on large arteries? J Hypertension 1999;17:701-705.

1. (canceled)

2. The method of claim 19, wherein the angiotensin II inhibitor is an angiotensin converting enzyme inhibitor.

3. The method of claim 2, wherein the angiotensin converting enzyme inhibitor is selected from the group consisting of captopril, enalapril, monopril, ramipril, cilazopril, fosinopril, perindopril, any other angiotensin converting enzyme inhibitor suitable for the treatment of hypertension, and combinations of any two or more thereof.

4. The method of claim 3, wherein the angiotensin converting enzyme inhibitor is captopril.

5. The method of claim 19, wherein the angiotensin II inhibitor is an angiotensin II receptor antagonist.

6. The method of claim 5, wherein the angiotensin II receptor antagonist is selected from the group consisting of eprosartan, candesartan, irbesartan, telmisartan, losartan, valsartan, any other angiotensin II receptor antagonist suitable for the treatment of hypertension, and combinations of any two or more thereof.

7. The method of claim 6, wherein the angiotensin II receptor antagonist is eprosartan.

8. The method of claim 19, wherein the nitric oxide donor is a long-acting nitrate or an extended release nitrate.

9. The method of claim 8, wherein the nitric oxide donor is selected from the group consisting of isosorbide mononitrate, isosorbide dinitrate, sinitrodil, any other nitric oxide donor suitable for the treatment of hypertension, and combinations of any two or more thereof.

10. The method of claim 9, wherein the nitric oxide donor is isosorbide mononitrate.

11. The method of claim 19, further comprising at least one of a range of drugs suitable for the treatment of hypertension selected from the group consisting of beta-blockers, calcium antagonists, diuretics, any other drugs suitable for the treatment of hypertension, and combinations of any two or more thereof.

12. The method of claim 11, wherein the beta-blockers are selected from the group consisting of atenolol, metoprolol, carvedilol, and combinations of any two or more thereof.

13. The method of claim 11, wherein the calcium antagonists are selected from the group consisting of nifedipine, amlodipine, felodipine, diltiazem, and combinations of any two or more thereof.

14. The method of claim 11, wherein the diuretics are selected from the group consisting of hydrochlorothiazide, amiloride, indapamide, chlorthalidone, frusemide, and combinations of any two or more thereof.

15. The method of claim 11, wherein the other drugs suitable for the treatment of hypertension are selected from the group consisting of clonidine, hydrallazine, methyl dopa, and combinations of any two or more thereof.

16. The method of claim 19 wherein the angiotensin II inhibitor is captopril and the nitric oxide donor is isosorbide mononitrate.

17. The method of claim 16, wherein the captopril is in a dose of 25 mg and the isosorbide mononitrate is in a dose of 60 mg.

18. The method of claim 19, wherein the hypertension to be treated and/or prevented is systolic hypertension of the elderly.

19. A method for the treatment and/or prevention of hypertension in a patient, said method comprising administering to the patient a therapeutically effective amount of a composition comprising a synergistic anti-hypertensive combination of:
a therapeutically effective amount of at least one angiotensin II inhibitor, and
a therapeutically effective amount of at least one nitric oxide donor;
said composition optionally further comprising a pharmaceutically acceptable carrier, diluent and/or adjuvent.

20. (canceled)

21. A method of treating and/or preventing hypertension in a patient, said method comprising administering to a patient a combination of:
a therapeutically effective amount of at least one angiotensin II inhibitor; and
a therapeutically effective amount of at least one nitric oxide donor,
wherein said combination has an synergistic anti-hypertensive effect on the patient.

22. The method of claim 21, wherein the angiotensin II inhibitor and the nitric oxide donor are administered simultaneously.

23-24. (canceled)
